(19) United States
(12) Patent Application Publication
(10) Pub. No.: US 2018/0014880 A1
(43) Pub. Date: Jan. 18, 2018

(54) ELECTROSURGICAL DEVICE FOR CHRONIC WOUND TREATMENT

(71) Applicant: Innoblative Designs, Inc., Chicago, IL (US)

(72) Inventors: Robert F. Rioux, Ashland, MA (US); Ryan M. Bean, Westminster, MA (US); Yearnchee C. Wang, Mill Creek, WA (US)

(21) Appl. No.: 15/646,697

(22) Filed: Jul. 11, 2017

Related U.S. Application Data
(60) Provisional application No. 62/361,138, filed on Jul. 12, 2016.

(51) Int. Cl. A61B 18/14; A61B 18/00

(52) U.S. Cl. CPC A61B 18/1492; A61B 2018/1467; A61B 2018/00011; A61B 2018/00577; A61B 2018/00589

(57) ABSTRACT

The present invention relates to an electrosurgical system including an electrosurgical device to be delivered to a wound site to provide chronic wound treatment. The device can be used during a wound care procedure to provide targeted energy at a wound site for reducing the accumulation of biofilm present and removing necrotic tissue and debris so as to promote, stimulate, and stabilize the wound healing process. The device can further be used during a surgical procedure, such as preparation for an orthopedic implant, in which the device is configured to selectively coagulate one or more pockets prepared within bone tissue for holding an implant so as to prevent or stop fluid accumulation (e.g., blood from vessel(s)) as a result of the implant preparation.

### CROSS-REFERENCE TO RELATED APPLICATIONS

This application claims the benefit of, and priority to, U.S. Provisional Application No. 62/361,138, filed Jul. 12, 2016, the content of which is hereby incorporated by reference herein in its entirety.

### FIELD

The present disclosure relates generally to medical devices, and, more particularly, to an electrosurgical device configured to provide targeted energy emission at a wound site for reducing the accumulation of biofilm present and removing necrotic tissue and debris so as to promote, stimulate, and stabilize the wound healing process.

### BACKGROUND

Wound healing is the body's natural response for repairing and regenerating dermal and epidermal tissue. The wound healing process is complex and fragile and may be susceptible to interruption or failure, especially in the instance of chronic wounds. A wound may be categorized as chronic if the wound does not heal in a predictable amount of time and in the orderly set of stages typical for wound healing. A number of factors may overwhelm the body's ability to effectively heal a wound, such as repeated trauma, continued pressure, an overriding illness, infection, or a restriction in blood supply to the wound area. More specifically, because the body's response to chronic wounds is often overwhelmed, the healing response becomes interrupted, resulting in instability and disorganization in the healing process.

Certain chronic wounds can be classified as ulcers of some type (i.e., diabetic ulcers, venous ulcers, and pressure ulcers). An ulcer is a break in a skin or a mucus membrane characterized by a loss of surface tissue, tissue disintegration, necrosis of epithelial tissue, nerve damage and pus. Venous ulcers typically occur in the legs and are thought to be attributable to either chronic venous insufficiency or a combination of arterial and venous insufficiency, resulting in improper blood flow or a restriction in blood flow that causes tissue damage leading to the wound. Pressure ulcers, commonly referred to as “bed sores,” are caused by ischemia that occurs when the pressure on the tissue is greater than the blood pressure in the capillaries at the wound site, thus restricting blood flow into the area. Accordingly, pressure ulcers typically occur in people with limited mobility or paralysis. For patients with long-standing diabetes and with poor glycemic control, a common condition is a diabetic foot ulcer (DFU), symptoms of which include slow healing surface lesions with peripheral neuropathy (which inhibits the perception of pain), arterial insufficiency, damage to small blood vessels, poor vascularization, ischemia of surrounding tissue, deformities, cellulitis tissue formation, high rates of infection and inflammation. Cellulitis tissue includes callous and fibrotic tissue. If left untreated, ulcers can become infected and gangrenous, which can result in disfiguring scars, deformity, and/or amputation of appendages having the affected tissue.

Additionally, several other types of wounds may progress to a chronic, non-healing condition. For example, surgical wounds at the site of incision may progress inappropriately to a chronic wound. Trauma wounds may similarly progress into chronic wound due to infection or involvement of other factors within the wound bed that inhibit proper healing. Burn treatment and related skin grafting procedures may also be compromised due to improper wound healing response and the presence of chronic wound formation conditions. In various types of burns, ulcers, and amputation wounds, skin grafting may be required. In certain instances, patients with ischemia or poor vascularity may experience difficulty in the graft “taking” resulting in the need for multiple costly skin grafting procedures. In patients where the risk of infection is high due to a weakened immune system (i.e., tissue impacted by radiation, patients undergoing cancer treatments, patients affected by immune compromised diseases such as HIV/AIDS), inflammation of a wound may be prolonged thereby interfering with the wound healing process and increasing the likelihood that the wound will become chronic, particularly where the wound site is unable to be sufficiently sterilized.

Current methods for the treatment of chronic wounds have shortcomings and thus fail to fully promote the wound healing process. For example, the moist, nutritionally supportive wound bed is an optimum environment for bacterial infection, particularly as a result of Staphylococcus aureus, the prevailing organism found in wounds. S. aureus and other bacteria secrete a protective self-surrounding matrix of extracellular polymeric substance (EPS), also known as a biofilm, which impairs the healing process. Healthcare providers (e.g., surgeons, clinicians, etc.) are limited in their arsenal to address such biofilms, due in part to their inherent antibiotic resistance. For example, a healthcare provider may turn to sharp debridement procedures via surgical, chemical, or mechanical means, for the removal of unviable tissue at the wound site in hopes of promoting the healing process. Although the removal of unviable tissue may result in the release of growth factors to promote healing, inevitably, within one or two days after initial debridement, the biofilm is able to reestablish itself and maintain a substantial pre-debridement antibiotic resistance. Furthermore, healthcare providers may perform debridement too aggressively in an attempt to effectively reduce bacterial biofilm, wherein such aggressive blind debridement may inadvertently remove healthy tissue and potentially expose vital structures, such as tendon and bone, and increase the severity of the wound.

### SUMMARY

The present invention relates to an electrosurgical system including an electrosurgical device to be delivered to a wound site to provide treatment of the wound. The device can be used during a wound care and treatment procedure to provide targeted energy emission at a wound site for coagulating biofilm and reducing the accumulation of such biofilm present within a wound bed so as to promote, stimulate, and stabilize the wound healing process. The device may further be used for the debridement of necrotic tissue and debris from the wound site and aspiration of such tissue and debris.

In particular, the device includes a probe generally acting as a handle and a deformable tip assembly extending from the probe and configured to provide radiofrequency (RF) treatment of the chronic wound tissue. The deformable tip assembly includes a nonconductive tip including a flexible body having a plurality of proximal ports and distal ports in communication with at least one lumen of the probe shaft. The flexible body includes a cavity configured to receive a conductive fluid, such as saline, from an irrigation source and further includes one or more perforations, which may include the proximal or distal ports, to allow the passage of the conductive fluid to an external surface of the flexible body. The deformable tip assembly further includes an electrode array including a plurality of conductive wires extending along an external surface of the nonconductive tip and configured to conduct energy to be carried by conductive fluid passing through the nonconductive tip.

The deformable tip assembly is configured to emit a non-ionizing radiation, such as radiofrequency (RF) energy in a bipolar configuration so as to treat a wound bed of the chronic wound tissue. More specifically, the nonconductive tip is flexible and configured to transition from a default state (e.g. generally spherical shape) to a deformed state (e.g., compressed sphere) upon a healthcare provider pressing the tip assembly against the wound bed. For example, the flexible body is configured to transition from a default state to a deformed state upon application of a compression force thereto and return to the default state upon removal of the compression force therefrom. When in the default state, the conductive wires are generally positioned a distance away from the wound bed sufficient to prevent the transmission of energy thereto. The compression of the nonconductive tip allows for the tip assembly to generally conform to the contour of the wound bed, allowing for improved contact and ablation/coagulation performance. The compression generally results in movement of a set of at least two conductive wires to come into contact with, or otherwise be positioned sufficiently adjacent to, a target portion of the wound bed to allow energy to be transmitted from the set of conductive wires to the wound bed by way of conductive fluid, thereby creating a virtual electrode for treating the chronic wound tissue. Accordingly, RF treatment of a target portion of the chronic wound tissue does not occur until the tip assembly is pressed against the desired target portion of the chronic wound tissue. The device further includes an aspiration lumen configured to be coupled to a vacuum source and provide suction of any debris or excess fluid during the treatment procedure.

Accordingly, the device of the present disclosure supports wound healing by providing a deformable applicator tip configured to generate a virtual electrode providing bipolar radiofrequency (RF) to a wound bed. The virtual electrode may be used to treat the chronic wound tissue in a variety of manners, including, but not limited to, debriding debris and necrotic tissue from the wound bed, coagulation of biofilm present within the wound bed to ultimately reduce the bacterial bioburden, removal of pathogens and bacteria from the wound bed, and hemostasis via coagulating of any underlying tissue so as to prevent or stop fluid accumulation (e.g., blood from vessels), each of which promotes, stimulates, and stabilizes the wound healing process.

The device of the present disclosure provides numerous advantages. The energy emitted from the virtual electrode of the applicator tip disrupts biological structures by creating ionic vibrations, which create friction and ultimately heat. The applicator tip is configured to desiccate the full thickness of biofilm present within a wound bed, which may be approximately 300 μm, while leaving underlying healthy tissue minimally damaged. At a cellular level, eradication of poly-microbial biofilm with a tolerable amount of healthy cell damage exposes remaining biofilm bacteria to the effect of the host immune system and antimicrobial agents. Furthermore, the device of the present disclosure is configured to provide chronic wound tissue treatment in a relative fast and efficient manner (e.g., within minutes), leading to minimal disruption in the current care path of wounds. At a clinical level, the device of the present disclosure may initially be used in conjunction with surgical or excisional debridement, as well as at the bedside on a post-procedure basis for outpatient maintenance therapy until the wound is healed. The device of the present disclosure is further useful in the pretreatment of wounds prior to excisional debridement, immediately following intraoperative surgical debridement, and as an adjunct to outpatient wound care therapy to prevent the re-establishment of biofilms. The device of the present disclosure has the potential to heal chronic, non-healing ulcers and dramatically improve patients' quality of life by avoiding many sequelae of lower extremity wounds and potential amputation.

It should be noted that the device of the present disclosure can further be used during a surgical procedure, such as preparation for an orthopedic implant, in which the device is configured to selectively coagulate one or more pockets prepared within bone tissue for holding an implant so as to prevent or stop fluid accumulation (e.g., blood from vessel(s)) as a result of the implant preparation.

In one aspect, the present disclosure provides a device for treating a chronic wound tissue. The device includes a probe comprising a nonconductive elongated shaft having a proximal end and a distal end and at least one lumen extending therethrough. The device further includes a nonconductive tip extending from the distal end of the probe shaft. The nonconductive tip includes a flexible body having a plurality of proximal ports and distal ports in communication with the at least one lumen of the probe shaft. The flexible body is configured to transition from a default state to a deformed state upon application of a compression force thereto and return to the default state upon removal of the compression force therefrom. For example, the flexible body of the nonconductive tip may include an elastomeric material or shape memory material. The nonconductive tip may include a substantially sphere-like shape when in the default state and a compressed shape when in the deformed state.

The device further includes electrode array including a plurality of independent conductive wires extending along an external surface of the nonconductive tip, wherein each of the plurality of wires passes through an associated one of the proximal ports and through a corresponding one of the proximal ports. Each of the plurality of wires, or one or more sets of a combination of wires, is configured to receive an electrical current to cause activation of one or more portions of the electrode array and conduct energy for at least one of ablation and coagulation of a target portion of the chronic wound tissue when the flexible body of the nonconductive tip is in the deformed state.

The chronic wound tissue includes a wound bed having at least one of necrotic tissue, bacteria, biofilm, and pathogens. Thus, upon receipt of electrical current from an energy source, at least one of the plurality of conductive wires is configured to conduct energy for ablation or coagulation of the biofilm in the wound bed.

In some embodiments, the flexible body of the nonconductive tip includes a cavity in fluid communication with at least one lumen of the probe shaft and configured to receive an amount of fluid delivered from the at least one lumen. The fluid delivered to the nonconductive tip may include a conductive fluid, such as saline. In some embodiments, delivery of fluid to the nonconductive tip may be controllable via a controller.

In some embodiments, the device further includes a heating element configured to heat the fluid within the cavity. The flexible body of the nonconductive tip may be configured to transfer the thermal energy from the heated fluid, held within the cavity of the body, to the target portion of the chronic wound tissue. The fluid may be heated to a temperature sufficient to cause necrosis of the target portion of the chronic wound tissue.

In some embodiments, the flexible body of the nonconductive tip may further include one or more perforations configured to allow passage of fluid from the cavity to an external surface of the flexible body. The one or more perforations may include, for example, at least one of the proximal and distal ports. Accordingly, energy conducted by each of the plurality of wires, or one or more sets of a combination of wires, may be carried by fluid passing through the one or more perforations for at least one of ablation and coagulation of the target portion of the chronic wound tissue. In some embodiments, the flexible body may be configured to release an amount of fluid through the one or more perforations in response to the compression force applied thereto. In some embodiments, an external surface of the flexible body of the nonconductive tip may include at least one portion of surface texturing to enhance fluid distribution. In some embodiments, the device further includes a sensor configured to detect the presence and/or absence of fluid on the external surface of the flexible body of the nonconductive tip. The ability to detect the presence or absence of fluid can be useful in determining the condition of the tissue being treated (e.g., whether the target portion has been sealed) or whether the device is functionally properly (e.g., fluid flow has stopped).

In some embodiments, when the flexible body of the nonconductive tip is in the default state, the electrode array is maintained a distance away from the target portion of the chronic wound tissue sufficient to prevent ablation or coagulation of the target portion. For example, the flexible body may include a distal tip portion configured to directly engage the target portion of the chronic wound tissue and maintain separation between the energy emitted from the electrode array and the target portion when the flexible body of the nonconductive tip is in the default state. The distal tip portion may be configured to compress inwardly to decrease distance between the electrode array and the target portion of the chronic wound tissue when the flexible body of the nonconductive tip transitions from the default state to the deformed state. When the flexible body of the nonconductive tip is in the deformed state, at least two of the plurality of conductive wires are positioned adjacent to the target portion of the chronic wound tissue to permit energy emitted from the electrode array to cause ablation or coagulation of the target portion.

In some embodiments, each of the plurality of conductive wires is independent from one another. Each of the plurality of conductive wires, or one or more sets of a combination of conductive wires, may be configured to independently receive an electrical current from an energy source and independently conduct energy. For example, in some embodiments, each of the plurality of wires is configured to convey energy away from the nonconductive tip upon receipt of the electrical current, wherein the energy includes RF energy. In some embodiments, each of the distal ports of the nonconductive tip corresponds to one proximal port such that a wire passing through corresponding distal and proximal ports extends along the length of the nonconductive tip. For example, in some embodiments, each of the plurality of wires extends through a different distal port. Additionally, or alternatively, each of the plurality of wires extends through a different proximal port. The plurality of wires may include at least two wires, wherein the wires axially translate along a longitudinal axis of the device.

In some embodiments, the device further includes a controller configured to selectively control supply of an electrical current to the electrode array in one or more operating modes. For example, the electrode array may be configured to operate in a bipolar mode, wherein a set of the plurality of conductive wires is configured to conduct RF energy. The controller may be configured to control one or more parameters associated with the supply of electrical current to the electrode array based on one or more operating modes. The one or more parameters may include, but are not limited to, the level of electrical current to be supplied, the length of time in which the electrical current is to be supplied, one or more intervals over which the electrical current is to be supplied, or a combination thereof.

In some embodiments, the device may further include a temperature sensor configured to sense a temperature of at least one of the energy transmitted from the one or more activated portions of the electrode array and the target portion of the chronic wound tissue during receipt of the energy transmitted from the one or more activated portions of the electrode array.

In some embodiments, the probe may include at least a second lumen extending through the elongated shaft and configured to be coupled to a vacuum source. Accordingly, the distal end of the probe shaft is in fluid communication with the vacuum source via the second lumen and, when the vacuum source is activated, the distal end is configured to provide suction so as to aspirate debris or fluid. Activation of the vacuum source may be controllable via a controller.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIGS. 1A and 1B are schematic illustrations of an electrosurgical system consistent with the present disclosure;

FIG. 2 is a perspective view of one embodiment of an electrosurgical device compatible with the system of FIG. 1A;

FIG. 3 is an enlarged view of the deformable tip assembly of the device of FIG. 2 in greater detail;

FIG. 4 is sectional view of the deformable tip assembly illustrating the nonconductive tip and the electrode array;

FIGS. 5A and 5B are perspective and side views illustrating placement of the deformable tip assembly to a wound bed of a chronic wound tissue while the nonconductive tip is in a default state;

FIGS. 6A and 6B are perspective and side views illustrating application of the of the deformable tip assembly against the wound bed of a chronic wound tissue resulting in transitioning of the deformable tip assembly from the default shape (shown in FIGS. 5A and 5B) to a deformed compressed shape upon pressing the tip assembly against the wound site;

FIG. 7 is a sectional view of the deformable tip assembly illustrating the nonconductive tip in the deformed state (compressed shape) and blocking of the aspiration lumen;

FIG. 8 is an enlarged view of the deformable tip assembly of the device of FIG. 2 illustrating additional components of the tip assembly consistent with the present disclosure;

FIG. 9 is an enlarged view of another embodiment of a deformable tip assembly compatible with the device of FIG. 2 in greater detail;

FIG. 10 is an exploded view of the deformable tip assembly of FIG. 9 illustrating the fluid retention member and electrode array separated from one another;

FIG. 11 is an enlarged view of the deformable tip assembly of FIG. 9 illustrating various capabilities, including emission of RF energy via the virtual electrode and suction via the aspiration lumen of the device; and

FIGS. 12A and 12B illustrate application of the of the deformable tip assembly of FIG. 9 to a wound bed of a chronic wound tissue and further transitioning of the deformable tip assembly from a default shape (shown in FIG. 12A) to a deformed compressed shape (shown in FIG. 12B) upon pressing the tip assembly against the wound site.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present disclosure is generally directed to an electrosurgical system including an electrosurgical device to be delivered to a wound site to provide treatment of the wound. The device can be used during a wound care and treatment procedure to provide targeted energy to a chronic wound tissue so as to promote, stimulate, and stabilize the wound healing process.

As used herein, chronic wound tissue generally refers to a wound that does not heal in an orderly set of stages and in a predictable amount of time. Wound healing is generally categorized into four stages: 1) clotting/hemostasis stage; 2) inflammatory stage; 3) tissue cell proliferation stage; and 4) tissue cell remodeling stage. Chronic wound tissue may include, but is not limited to, wound tissue attributable to diabetic ulcers, venous ulcers, pressure ulcers, surgical wounds, trauma wounds, burns, amputation wounds, radiated tissue, tissue affected by chemotherapy treatment, and infected tissue compromised by a weakened immune system.

Although the following description focuses on the ability of the device to treat chronic wound tissue, it should be noted that the device of the present disclosure can further be used during a surgical procedure, such as preparation for an orthopedic implant, in which the device is configured to selectively coagulate one or more pockets prepared within bone tissue for holding an implant so as to prevent or stop fluid accumulation (e.g., blood from vessel(s)) as a result of the implant preparation.

Generally, the device includes a probe acting as a handle and a deformable tip assembly extending from the probe and configured to provide radiofrequency (RF) treatment of the chronic wound tissue. The deformable tip assembly includes a nonconductive tip including a flexible body having a plurality of proximal ports and distal ports in communication with at least one lumen of the probe shaft. The flexible body includes a cavity configured to receive a conductive fluid, such as saline, from an irrigation source and further includes one or more perforations, which may include the proximal or distal ports, to allow the passage of the conductive fluid to an external surface of the flexible body. The deformable tip assembly further includes an electrode array including a plurality of conductive wires extending along an external surface of the nonconductive tip and configured to conduct energy to be carried by conductive fluid passing through the nonconductive tip.

The deformable tip assembly is configured to emit a non-ionizing radiation, such as radiofrequency (RF) energy in a bipolar configuration so as to treat a wound bed of the chronic wound tissue. More specifically, the nonconductive tip is flexible and configured to transition from a default state (e.g. generally spherical shape) to a deformed state (e.g., compressed sphere) upon a healthcare provider pressing the tip assembly against the wound bed. For example, the flexible body is configured to transition from a default state to a deformed state upon application of a compression force thereto and return to the default state upon removal of the compression force therefrom. When in the default state, the conductive wires are generally positioned a distance away from the wound bed sufficient to prevent the transmission of energy thereto. The compression of the nonconductive tip allows for the tip assembly to generally conform to the contour of the wound bed, allowing for improved contact and ablation/coagulation performance. The compression generally results in movement of a set of at least two conductive wires to come into contact with, or otherwise be positioned sufficiently adjacent to, a target portion of the wound bed to allow energy to be transmitted from the set of conductive wires to the wound bed by way of conductive fluid, thereby creating a virtual electrode for treating the chronic wound tissue. Accordingly, RF treatment of a target portion of the chronic wound tissue does not occur until the tip assembly is pressed against the desired target portion of the chronic wound tissue. The device further includes an aspiration lumen configured to be coupled to a vacuum source and provide suction of any debris or excess fluid during the treatment procedure.

Accordingly, the device of the present disclosure supports wound healing by providing a deformable applicator tip configured to generate a virtual electrode providing bipolar radiofrequency (RF) to a wound bed. The virtual electrode may be used to treat the chronic wound tissue in a variety of manners, including, but not limited to, debriding debris and necrotic tissue from the wound bed, coagulation of biofilm present within the wound bed to ultimately reduce the bacterial bioburden, removal of pathogens and bacteria from the wound bed, and hemostasis via coagulating of any underlying tissue so as to prevent or stop fluid accumulation (e.g., blood from vessels), each of which promotes, stimulates, and stabilizes the wound healing process.

The device of the present disclosure provides numerous advantages. The energy emitted from the virtual electrode of the applicator tip disrupts biological structures by creating ionic vibrations, which create friction and ultimately heat. The applicator tip is configured to desiccate the full thickness of biofilm present within a wound bed, which may be approximately 300 μm, while leaving underlying healthy tissue minimally damaged. At a cellular level, eradication of poly-microbial biofilm with a tolerable amount of healthy cell damage exposes remaining biofilm bacteria to the effect of the host immune system and antimicrobial agents. Furthermore, the device of the present disclosure is configured to provide chronic wound tissue treatment in a relative fast and efficient manner (e.g., within minutes), leading to minimal disruption in the current care path of wounds. At a clinical level, the device of the present disclosure may initially be used in conjunction with surgical or excisional debridement, as well as at the bedside on a post-procedure basis for outpatient maintenance therapy until the wound is healed. The device of the present disclosure is further useful in the pretreatment of wounds prior to excisional debridement, immediately following intraoperative surgical debridement, and as an adjunct to outpatient wound care therapy to prevent the re-establishment of biofilms. The device of the present disclosure has the potential to heal chronic, non-healing ulcers and dramatically improve patients' quality of life by avoiding many sequelae of lower extremity wounds and potential amputation.

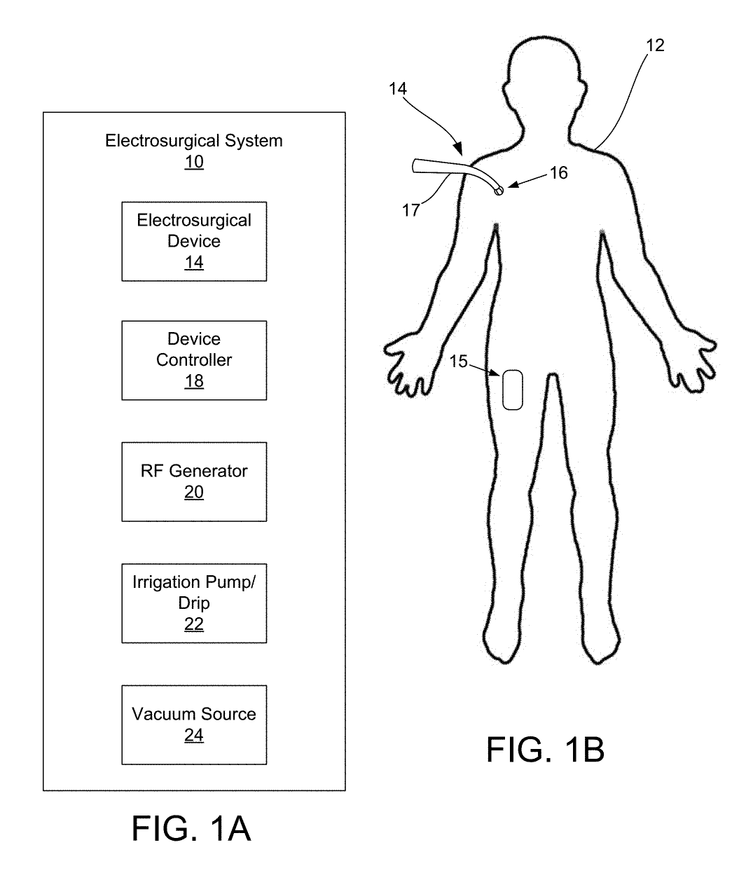

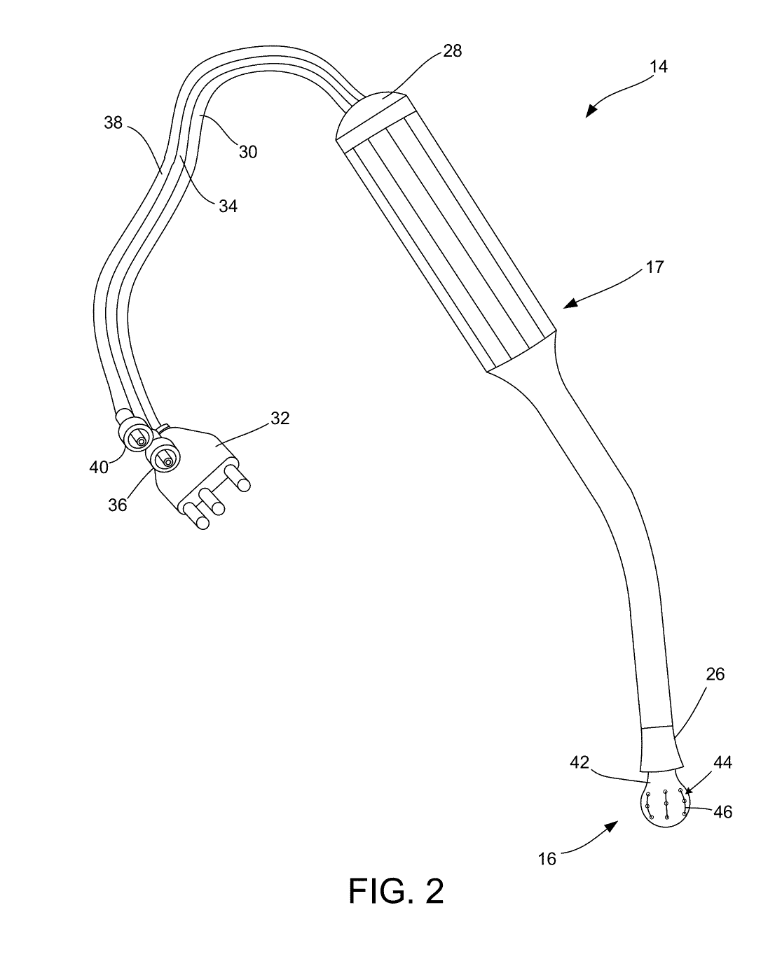

FIGS. 1A and 1B are schematic illustrations of an electrosurgical system 10 for providing improved wound care treatment for a patient 12. The electrosurgical system 10 generally includes an electrosurgical device 14, which includes a probe having a deformable tip assembly 16 and an elongated catheter shaft 17 to which the tip assembly 16 is coupled. The catheter shaft 17 may generally include a nonconductive elongated member including a fluid delivery lumen and an aspiration lumen, as will be described in greater detail herein. The electrosurgical device 14 may further be coupled to a device controller 18, a radiofrequency (RF) generator 20 over an electrical connection (electrical line 30 shown in FIG. 2), an irrigation pump or drip 22 over a fluid connection (fluid line 34 shown in FIG. 2), and a vacuum source 24 over a connection (connection line 38 shown in FIG. 2).

The device controller 18 may include hardware/software configured to provide a user with the ability to control electrical output to the electrosurgical device 14 in a manner so as to control ablation output to a wound site for treating chronic wound tissue. For example, as will be described in greater detail herein, the electrosurgical device may be configured to operate at least in a “bipolar mode” based on input from a user (e.g., surgeon, clinician, etc.) resulting in the emission of radiofrequency (RF) energy in a bipolar configuration. In some embodiments, the device 14 may be configured to operate in other modes, such as a “measurement mode”, in which data can be collected, such as certain measurements (e.g., temperature, conductivity (impedance), etc.) that can be taken and further used by the controller 18 so as to provide an estimation of the state of tissue during a wound treatment procedure, as will be described in greater detail herein.

Further still, the device controller 18 may include a custom ablation shaping (CAS) system configured to provide a user with custom ablation shaping, which includes the creation of custom, user-defined ablation geometries or profiles from the electrosurgical device 14. The CAS system may further be configured to provide ablation status mapping based on real-time data collection (e.g., measurements) collected by the device, wherein such a CAS system is described in co-pending U.S. Provisional Application Ser. No. 62/290,108, filed Feb. 2, 2016. In some cases, the device controller 18 may be housed within the electrosurgical device 14. The ablation generator 20 may also be connected to a separate return electrode 15 that is attached to the skin of the patient 12.

As will be described in greater detail herein, during a chronic wound tissue treatment procedure, the generator 20 may generally provide RF energy (e.g., electrical energy in the radiofrequency (RF) range (e.g., 350-800 kHz)) to an electrode array of the electrosurgical device 14, as controlled by the device controller 18. At the same time, saline may also be provided to and released from the tip assembly 16. In some The RF energy travels through the blood and tissue of the patient 12 to a return electrode and, in the process, provides ablation the region(s) of tissue adjacent to portions of the electrode array that have been activated.

FIG. 2 is a perspective view of electrosurgical device 14. As previously described, the electrosurgical device 14 includes a probe 17 including an elongated shaft configured as a handle and adapted for manual manipulation. Accordingly, as shown in FIG. 2, the probe 17 is in the form of a handle having a distal end 26 to which the tip assembly 16 is coupled and a proximal end 28. The probe 17 may generally resemble a Yankauer handle, for example. As shown, the proximal end 28 of the probe 17 may be coupled to the generator 20, the irrigation pump 22, and the vacuum source 24 via connection lines or fittings. For example, the probe 17 is coupled to the generator 20 via an electrical line 30, coupled to the irrigation pump 22 via a fluid line 34, and coupled to the vacuum source via a connection line 38. Each of the electrical line 30, fluid line 34, and connection line 38 may include an adaptor end 32, 36, 40 configured to couple the associated lines with a respective interface on the generator 20, irrigation pump 22, and vacuum source.

In some examples, the electrosurgical device 14 may further include a user interface (not shown) serving as the device controller 18 and in electrical communication with at least one of the generator 20, the irrigation pump 22, and/or vacuum source 24, and the electrosurgical device 14. The user interface 28 may include, for example, selectable buttons for providing an operator with one or more operating modes with respect to controlling the energy emission output of the device 14, as will be described in greater detail herein. For example, selectable buttons may allow a user to control electrical output to the electrosurgical device 14 in a manner so as to control the coagulation or debridement of portions of a wound bed on the chronic wound tissue. Furthermore, in some embodiments, selectable buttons may provide an operator to control the delivery of fluid from the irrigation pump 22 and/or activation of the vacuum source 24 to control suction at the distal end 26 of the probe 17.

The tip assembly 16 includes a nonconductive tip 42 extending from the distal end 26 of the probe shaft 17 and an electrode array 44 comprising a plurality of independent conductive wires 46 extending along an external surface of the nonconductive tip 42. As will be described in greater detail herein, the tip assembly 16 is deformable, in that, during treatment, an operator may apply RF energy from the tip assembly 16 to a desired portion of a wound by simply pressing the tip assembly 16 against the wound site so as to coagulate, debride, or otherwise remove necrotic tissue, debris, biofilm, bacteria, or the like. More specifically, the nonconductive tip 42 and electrode array generally flexible and configured to transition from default shapes (e.g. generally spherical) to deformed shapes (e.g., compressed spheres) upon a healthcare provider pressing the tip assembly 16 against the wound bed. The compression of the deformable tip assembly 16 allows for the tip assembly to conform to the contour of the wound bed, allowing for improved contact and ablation/coagulation performance.

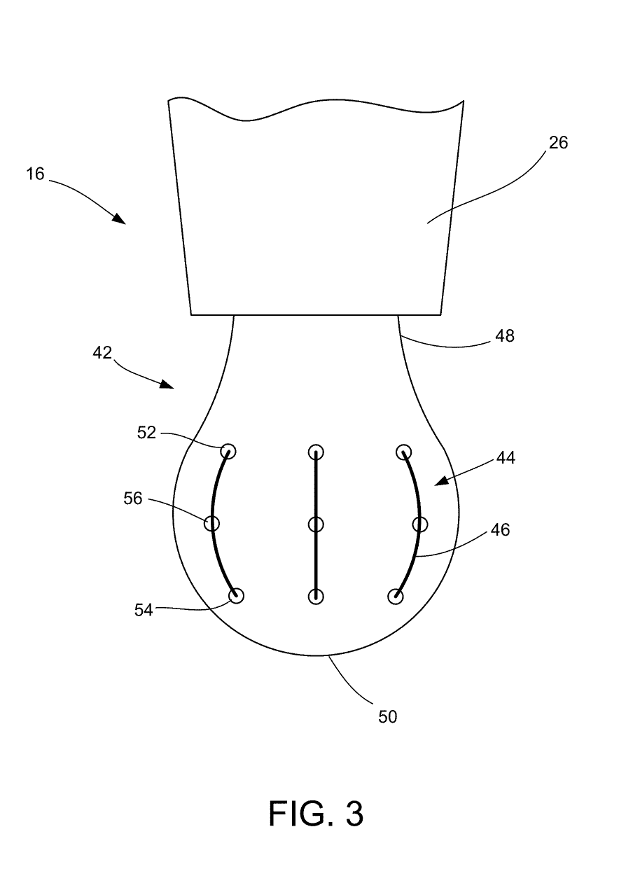

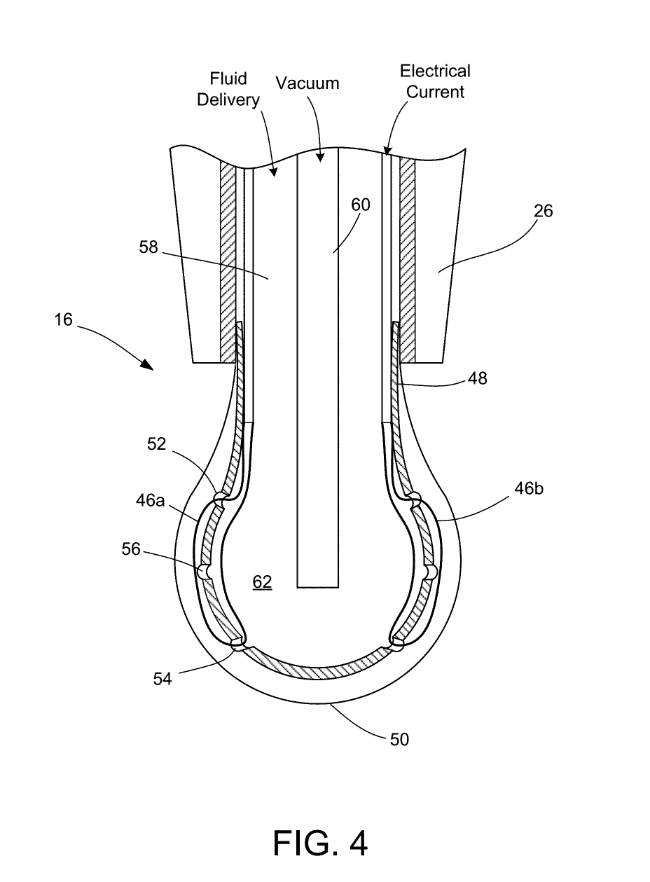

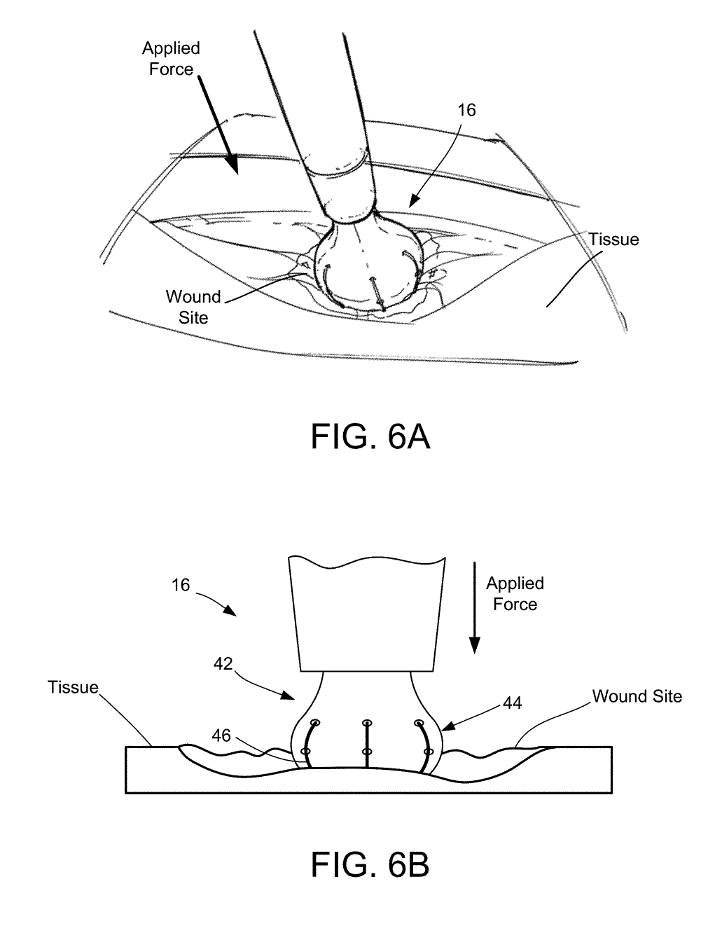

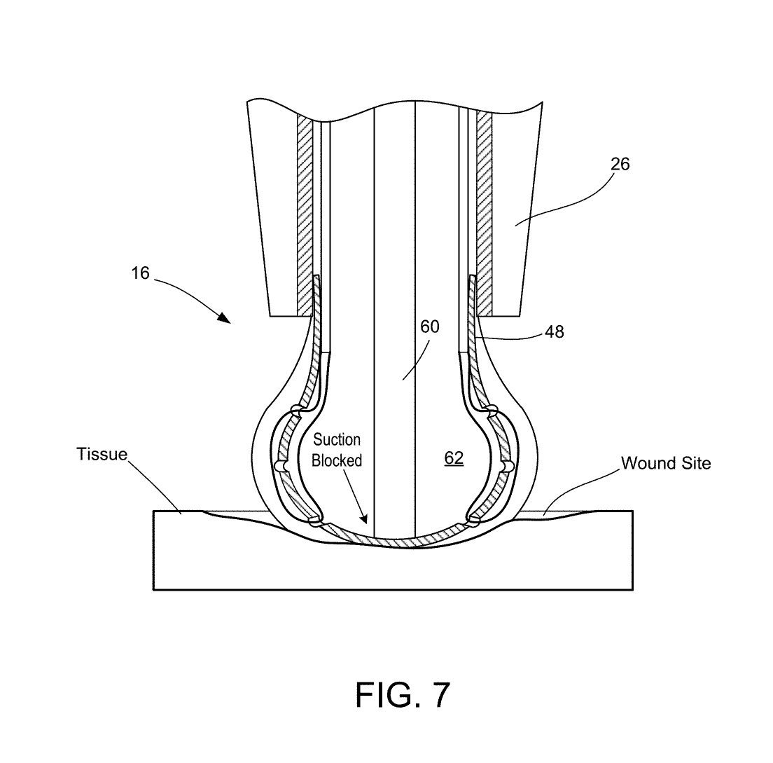

FIG. 3 is an enlarged view of the deformable tip assembly 16 and FIG. 4 is sectional view of the deformable tip assembly 16 illustrating the nonconductive tip and the electrode array relative to one another. As shown, the nonconductive tip 42 includes a proximal end 48 coupled to the distal end 26 of the probe shaft 17 and a distal end 50. As will be described in greater detail herein, the nonconductive tip 42 includes a flexible body configured to transition from a default state to a deformed state upon application of a compression force thereto and return to the default state upon removal of the compression force therefrom. Accordingly, the nonconductive tip 42 may include an elastomeric or shape memory material. As shown in FIGS. 3 and 4, the nonconductive tip 42 has a generally spherical shape when in the default state. Upon application of a force (e.g., pressing of the tip 42 against a wound bed or the like), the nonconductive tip 42 is configured to flex and transition into a deformed state, where portions of the nonconductive tip 42 can become deformed such that nonconductive tip assumes a compressed shape (shown in FIGS. 6B and 7).

As shown in FIGS. 3 and 4, the nonconductive tip 42 includes plurality of proximal ports 52 and distal ports 54 in communication with the at least one lumen of the probe shaft 17. The proximal ports 52 and distal ports 54 generally serve as openings through which conductive wires 46 of the electrode array 44 may pass. For example, each of the plurality of wires 46 passes through an associated one of the proximal ports and through a corresponding one of the proximal ports. Accordingly, the number of proximal ports 52 and distal ports 54 may generally be equal to the number of conductive wires 46, such that each conductive wire 46 can extend through a different distal port 54, which allows the conductive wires 46 to remain electrically isolated from one another. In other examples, one or more conductive wires can extend through the same distal port 54. The nonconductive tip 42 may further include one or more perforations 56 configured to allow passage of fluid from the within the nonconductive tip 42 to an external surface of the nonconductive tip 42, as will be described in greater detail herein.

Upon passing through a distal port 54, each conductive wire 46 can extend along an external surface of the nonconductive tip 42. In some examples, the length of the conductive wire 46 extending along the external surface is at least 20% (e.g., at least, 50%, 60%, 75%, 85%, 90%, or 99%) of the length of the nonconductive tip 42. The conductive wire 46 can then re-enter the nonconductive tip 42 through a corresponding proximal port 52. For example, as shown in FIG. 4, conductive wire 46a passes through distal port 54, extends along a length of the external surface of the nonconductive tip 42, and passes through an associated proximal port 52 and into a cavity of the nonconductive tip 42, while conductive wire 46b is electrically isolated from conductive wire 46a in that it passes through its own associated proximal and distal ports. The wires 46 are configured to receive energy in the form of electrical current from the RF generator 20 and emit RF energy in response. The conductive wires 46 can be formed of any suitable conductive material (e.g., a metal such as stainless steel, nitinol, or aluminum).

As shown, one or more of the conductive wires 46 can be electrically isolated from one or more of the remaining conductive wires, such that the electrical isolation enables various operation modes for the electrosurgical device 14. For example, electrical current may be supplied to one or more conductive wires in a bipolar mode, a unipolar mode, or a combination bipolar and unipolar mode. In the unipolar mode, ablation energy is delivered between one or more conductive wires of the electrode array 44 and a return electrode 15, for example. In bipolar mode, energy is delivered between at least two of the conductive wires, while at least one conductive wire remains neutral. In other words, at least, one conductive wire functions as a grounded conductive wire (e.g., electrode) by not delivering energy over at least one conductive wire.

Since each conductive wire 46 in the electrode array 44 is electrically independent, each conductive wire 46 can be connected in a fashion that allows for impedance measurements using bipolar impedance measurement circuits. For example, the conductive wires can be configured in such a fashion that tetrapolar or guarded tetrapolar electrode configurations can be used. For instance, one pair of conductive wires could function as the current driver and the current return, while another pair of conductive wires could function as a voltage measurement pair. Accordingly, a dispersive ground pad can function as current return and voltage references. Their placement dictate the current paths and thus having multiple references can also benefit by providing additional paths for determining the ablation status of the tissue. The impedance measurement capability of the device is described in co-pending U.S. Provisional Application No. 62/248,157 filed on Nov. 10, 2015, U.S. Provisional Application No. 62/275,984 filed on Jan. 7, 2016, and U.S. Provisional Application Ser. No. 62/290,108, filed Feb. 2, 2016, the entireties of which are incorporated by reference.

As previously described, in some embodiments, energy conducted by one or more of the wires 46 is carried by the fluid weeping from the nonconductive tip 42, thereby creating a virtual electrode for treating the chronic wound tissue. For example, the nonconductive tip 42 is configured to receive and retain an amount of fluid delivered from at least one lumen of the probe shaft 17. As shown in FIG. 4, the probe shaft 17 may include a fluid lumen 58 coupled to the irrigation pump 22 via the fluid line 34 and configured to receive fluid therefrom. The nonconductive tip 42 further includes a cavity 60 in fluid communication with the fluid lumen 58 of the probe shaft 17 and configured to receive an amount of fluid delivered from lumen 58. The fluid delivered to the nonconductive tip 42 may include a conductive fluid, such as saline. The saline within the cavity 60 may be distributed to an external surface of the tip 42 through the one or more perforations 56 and/or the ports (e.g., to the proximal ports 52 and distal ports 54). The saline weeping through the perforations 56 and/or ports 52, 54 to an outer surface of the nonconductive tip 42 is able to carry electrical current from the electrode array 44, such that energy is transmitted from the electrode array 44 to a target portion of the chronic wound tissue by way of the saline, thereby creating a virtual electrode. Accordingly, upon fluid weeping through the perforations and/or ports, a pool or thin film of fluid is formed on the exterior surface of the nonconductive tip 42 and configured to ablate and/or coagulate via the electrical current conducted by the one or more conductive wires 46 of the electrode array 42.

The probe shaft 17 may further include an aspiration lumen 62 configured to be coupled to the vacuum source 24 via the connection line 38. Accordingly, the distal end 26 of the probe shaft 17 is in fluid communication with the vacuum source 24 via the aspiration lumen, such that, when the vacuum source 24 is activated, the distal end 26 is configured to provide suction so as to aspirate debris or fluid during the treatment procedure. As will be described in greater detail herein, when the nonconductive tip 42 is in the default state, the tip assembly 16 may allow for suction, while suction may be prevented when the nonconductive tip 42 is in the deformed state.

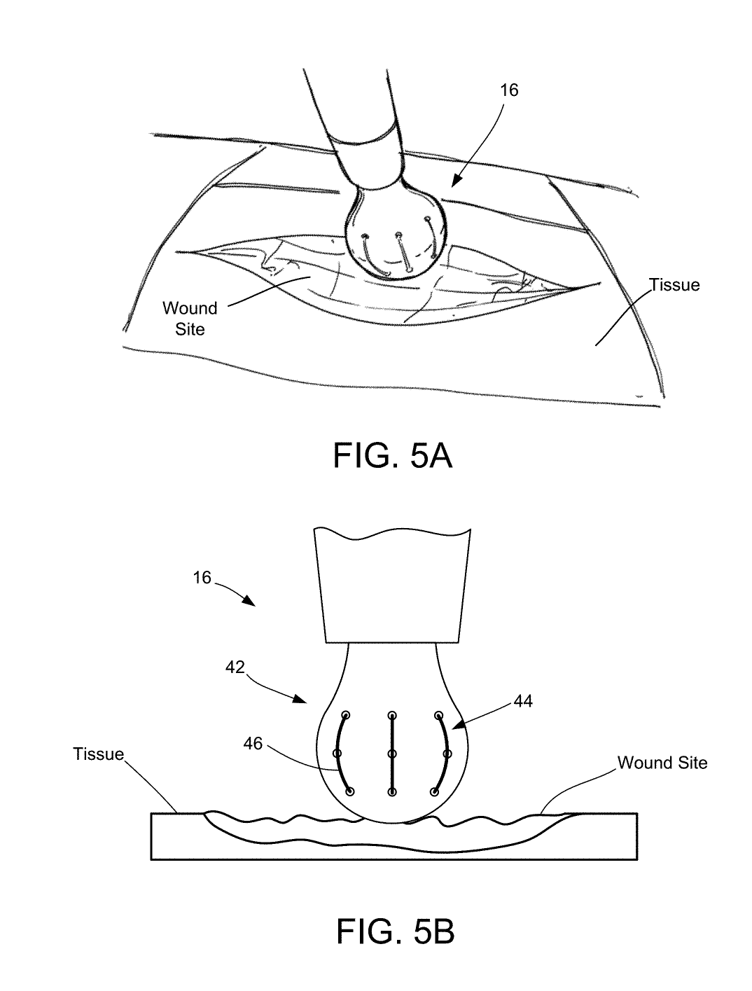

FIGS. 5A and 5B are perspective and side views illustrating placement of the deformable tip assembly 16 to a wound bed of a chronic wound tissue while the nonconductive tip 42 is in a default state. As shown, the nonconductive tip 42 has a generally spherical shape when in the default state. It should be noted, however, that the nonconductive tip 42 may include a variety of shapes or dimensions when in the default shape and is not limited to a spherical shape. When the nonconductive tip 42 is in the default state, the electrode array 44 is maintained a distance away from the target portion of the chronic wound tissue sufficient to prevent ablation or coagulation of the target portion. For example, the distal end 50 of the nonconductive tip 42 is configured to directly engage the target portion of the chronic wound tissue and maintain separation between the electrode array 44 and the target portion of the chronic wound tissue. This particular configuration allows for a healthcare provider to place the tip assembly 16 in a desired position prior to commencing the transmission of energy to the target portion of the wound. When the healthcare provider is satisfied with the positioning of the tip assembly 16, they need only press the tip assembly 16 against the target portion of the wound bed, which in turn results in transitioning of the nonconductive tip 42 from the default state to the deformed state to allow for transmission of RF energy to the target portion.

For example, FIGS. 6A and 6B are perspective and side views illustrating application of the of the deformable tip assembly 16 against the wound bed of a chronic wound tissue resulting in transitioning of the nonconductive tip 42 from the default shape to the deformed state. As shown, the distal tip portion may be configured to compress inwardly to decrease distance between the electrode array 44 and the target portion of the chronic wound tissue when the flexible body of the nonconductive tip 42 transitions from the default state to the deformed state. For example, when the flexible body of the nonconductive tip 42 is in the deformed state, at least two of the conductive wires 46 are positioned adjacent to the target portion of the chronic wound tissue to permit energy emitted from the electrode array 42 to cause ablation or coagulation of the target portion. In particular, the compression generally decreases the distance between the conductive wires 46 and the wound bed, which may allow for direct contact between the targeted portion and the conductive wires 46 and/or direct contact between the saline fluid weeping through the perforations 56 and/or ports 52, 54 and carrying energy from the conductive wires 56. Accordingly, a healthcare provide need only press the tip 42 against the wound site when the electrical current is so as to cause coagulation, debridement, or otherwise remove necrotic tissue, debris, biofilm, bacteria, or the like. Upon removing the tip 42 from the wound, the tip 42 may be configured to return to the default state and allow subsequent passes at the wound. The compression of the deformable tip assembly 16 allows for the tip assembly 16 to generally conform to the contour of the wound bed, allowing for improved contact and ablation and/or coagulation performance.

FIG. 7 is a sectional view of the deformable tip assembly 16 illustrating the nonconductive tip in the deformed state (compressed shape) and blocking of the aspiration lumen 62. As previously described, the probe shaft 17 further includes an aspiration lumen 62 configured to be coupled to the vacuum source 24 via the connection line 38 and allow for suction of debris or fluid during the treatment procedure. However, upon transition of the nonconductive tip 42 to the deformed state, an opening of the aspiration lumen 62 may be blocked, thereby preventing suction. When the compression force is release (i.e., when the tip assembly 16 is move away from the wound), the opening of the aspiration lumen 62 may be cleared and suction may resume.

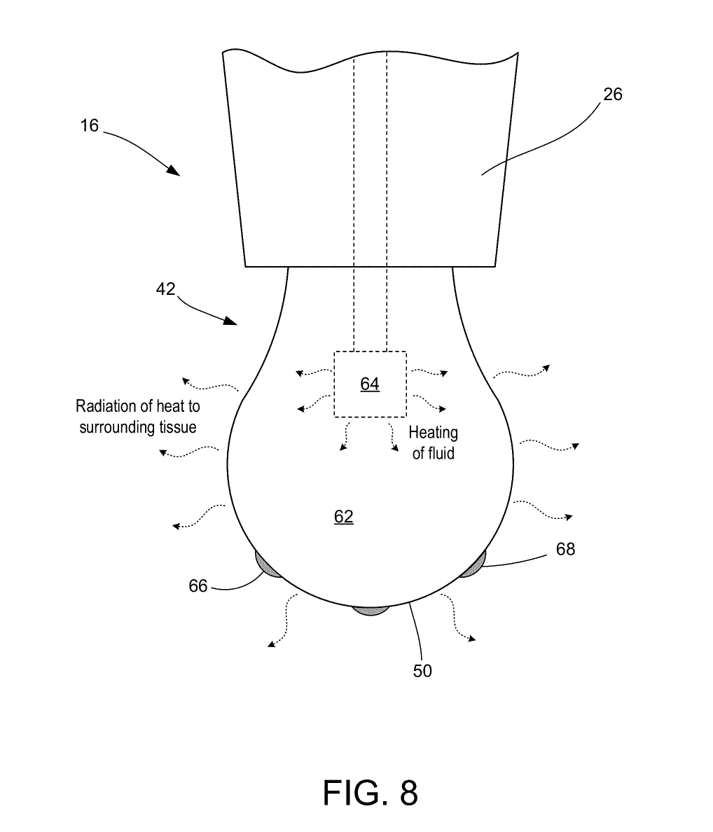

FIG. 8 is an enlarged view of the deformable tip assembly 16 illustrating additional components consistent with the present disclosure. For example, the device 14 may further include a heating element 64 configured to heat fluid within the cavity 62 of the nonconductive tip 42. The heating element 64 may be configured to receive an electrical current from a source (e.g., from an external source, such as the RF generator 20) and generate thermal energy which, in turn, may heat up the fluid within the cavity 62. The nonconductive tip 42 may be configured to transfer the thermal energy from the heat fluid within the cavity to the target portion of the chronic wound tissue, such that, upon making physical contact between the external surface of the nonconductive tip 42 with the target portion, thermal energy is provided to the target portion. The heating element 64 is configured to heat the fluid to a temperature sufficient to cause necrosis of the target portion of chronic wound tissue. For example, in some embodiments, the heating element 64 may be configured to heat the fluid to a temperature between 30° C. and 100° C. In some embodiments, the heating element 64 may be configured to heat the fluid to a temperature between approximately 80° C. and 97° C. It should be noted that, in addition to providing RF energy via the virtual electrode arrangement, the device 14 of the present disclosure is configured to provide transmission of thermal energy to the target portion via the heated fluid configuration described herein.

The operation of the heating element 64 may be controlled via the controller 18. For example, in some embodiments, the controller 18 may be used to control the supply of electrical current to the heating element 64 and further control the amount of thermal energy conducted by the heating element 64, thereby providing control of the temperature of the fluid.

As previously described, the device 14 may be configured to operate in other modes, such as a “measurement mode”, in which data can be collected, such as certain measurements (e.g., temperature, conductivity (impedance), etc.) that can be taken and further used by the controller 18 so as to provide an estimation of the state of tissue during a wound treatment procedure. As shown in FIG. 8, the device 14 may include one or more sensors for detecting certain properties or characteristics during operation of the device 14. For example, in some embodiments, the device 14 may further include a sensor 66 configured to detect the presence and/or absence of fluid on an external surface of the nonconductive tip 42. The sensor 66 may be positioned along the external surface of the tip 42. The data associated with the detected presence or absence of fluid may be provided to the controller 18 to be used for determining the condition of the tissue being treated (e.g., whether the target portion has been sealed) or whether the device is functionally properly (e.g., fluid flow has stopped). The device 14 may further include a temperature sensor 68 configured sense a temperature associated with a component of the device 14 or the target portion of chronic wound tissue during a procedure. For example, the temperature sensor 68 may be configured to sense the temperature of the energy transmitted from the one or more activated portions of the electrode array 44, the temperature of the heating element 64, the temperature of heated fluid within the cavity 62 of the nonconductive tip 42, the temperature of target portion of the chronic wound tissue during a treatment procedure, and a combination thereof. The data associated with detected temperatures from the temperature sensor 68 maybe provided to the controller 18 to be used for controlling operating parameters of the device 14 (e.g., increasing or decreasing energy output from tip assembly to maintain operation within appropriate ranges for desired operating mode) as well as providing an operator with an estimation of the state of the target portion of the chronic wound tissue.

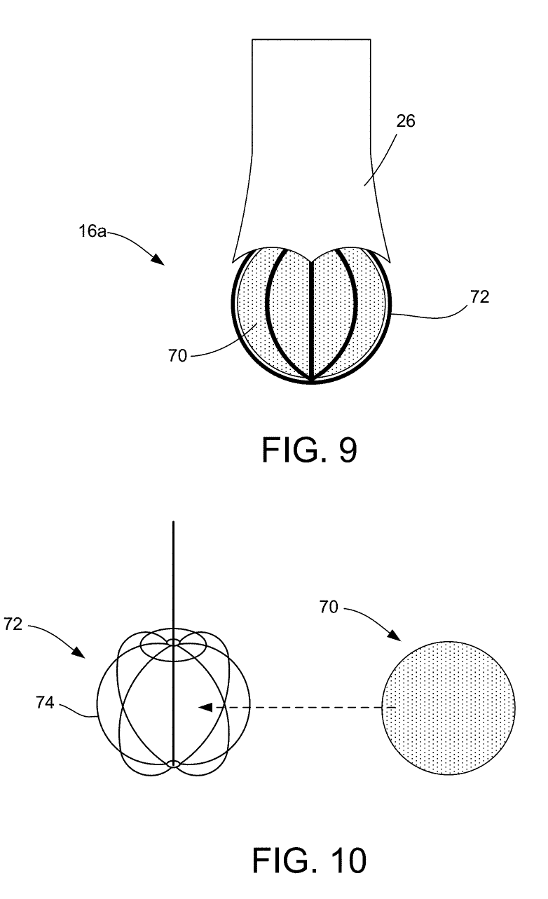

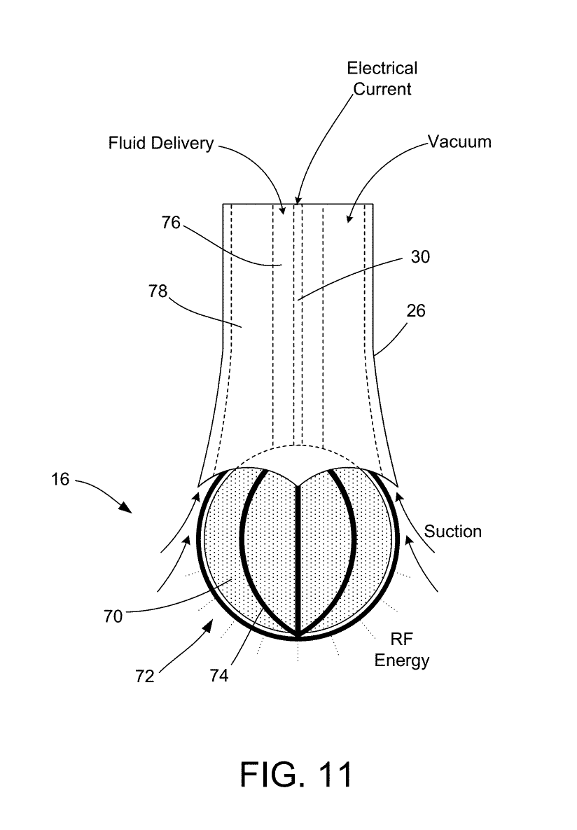

FIGS. 9-12B depict another embodiment of a deformable tip assembly 16a compatible with the electrosurgical device 14 and system 10 of the present disclosure. FIG. 9 is an enlarged view of the deformable tip assembly 16a. As shown, the tip assembly 16a includes a fluid retention member 70 configured to receive a conductive fluid from an irrigation source and an electrode array 72 surrounding the fluid retention member configured to conduct energy. As shown in FIG. 10, the fluid retention member 70 generally includes a flexible porous body configured to receive and retain an amount of fluid delivered from at least one lumen of the probe shaft 17. As shown in FIG. 11, for example, the probe 17 may include at least a fluid lumen 76 coupled to the irrigation pump 22 via the fluid line 34 and configured to receive fluid therefrom. The porous body may generally include a plurality of partially open cells configured to retain an amount of fluid within. In some embodiments, the fluid retention member 70 is made from a sponge-like material. The porous body is flexible in that it is configured to transition from a default shape (e.g., generally spherical) to a deformed shape (e.g., non-spherical) upon a compression force applied thereto. In other words, the porous body can be squeezed into a smaller volume and then, upon removal of the compression force, return to the default shape. In the event that the porous body has fluid retained therein, compression of the fluid retention member 70 results in an amount of fluid to be released. As will be described in greater detail herein, the release of fluid from the fluid retention member 70 allows for the creation of a virtual electrode on the exterior of the tip assembly 16a. In particular, the released fluid is configured to carry energy conducted by the electrode array 72 to a desired portion of the wound site. Accordingly, the fluid is generally a conductive fluid, such as saline.

The electrode array 72 is composed of a plurality of conductive wires 74 surrounding the fluid retention member 70. The wires 74 are configured to receive energy in the form of electrical current from the RF generator 20 and emit RF energy in response. As previously described, in some embodiments, energy conducted by one or more of the wires 74 is carried by the fluid released from the fluid retention member 70, thereby creating a virtual electrode for treating the chronic wound tissue. The conductive wires 74 can be formed of any suitable conductive material (e.g., a metal such as stainless steel, nitinol, or aluminum).

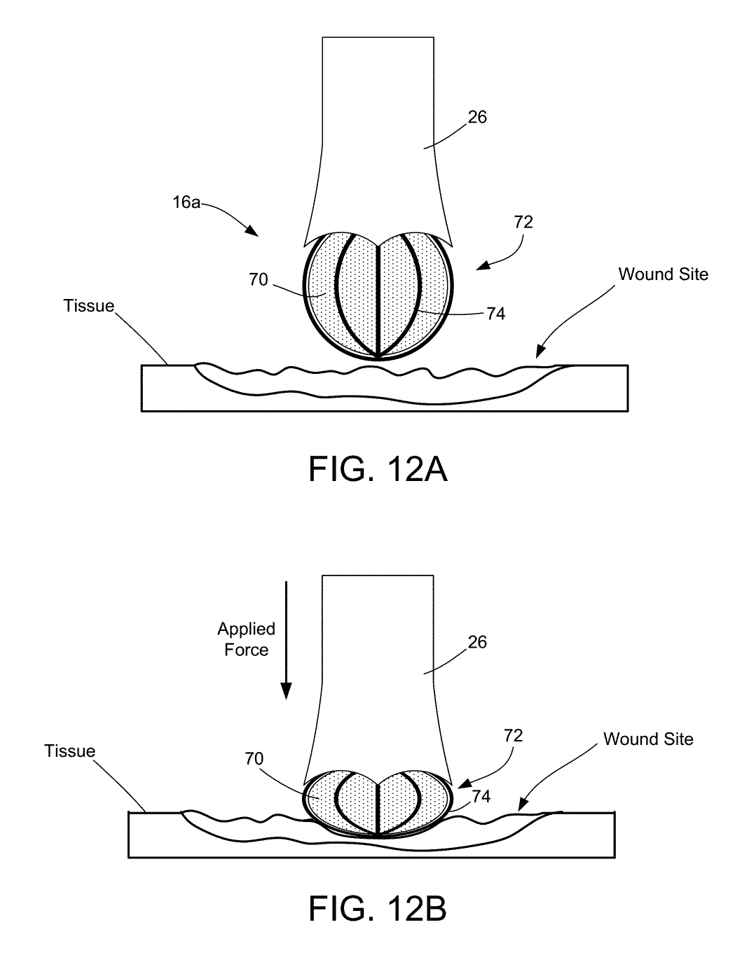

As shown, the plurality of wires 74 generally forms a sphere-like cage surrounding the fluid retention member 70. Similar to the fluid retention member 70, each of the conductive wires 74 is flexible, such that each wire 74 is configured to transition from a default shape to a deformed shape upon a compression force applied thereto. Accordingly, the wires 74 may include a shape memory material, such as nitinol, for example. Thus, the electrode array may transition from a default sphere-like shape to a compressed shaped when the tip assembly 16a is pressed against a desired portion of a wound site, for example (as shown in FIG. 12B and described in greater detail herein).

As shown in FIG. 11, the probe shaft 17 further includes an aspiration lumen 78 configured to be coupled to the vacuum source 24 via the connection line 38. Accordingly, the distal end 26 of the probe shaft 17 is in fluid communication with the vacuum source 24 via the aspiration lumen, such that, when the vacuum source 24 is activated, the distal end 26 is configured to provide suction so as to aspirate debris or fluid during the treatment procedure.

FIGS. 12A and 12B illustrate application of the of the deformable tip assembly 16a to a wound bed of a chronic wound tissue and further transitioning of the deformable tip assembly from a default shape (shown in FIG. 12A) to a deformed compressed shape (shown in FIG. 12B) upon pressing the tip assembly 16a against the wound site.

As previously described, both the electrode array 72 and the fluid retention member 70 are flexible and configured to transition from default shapes (e.g. generally spherical) to deformed shapes (e.g., compressed spheres) upon a healthcare provider pressing the deformable tip assembly 16a against a wound site (e.g., wound bed of a chronic wound tissue). The compression of the deformable tip assembly 16a allows for the tip assembly 16a to generally conform to the contour of the wound bed, allowing for improved contact and ablation/coagulation performance. The compression results in the release of conductive fluid from the fluid retention member 70 such that energy is transmitted from the electrode array 72 to the wound bed by way of the released conductive fluid, thereby creating a virtual electrode for treating the chronic wound tissue. In particular, the deformable tip assembly 16a is configured to generate virtual electrode providing bipolar radiofrequency ablation (RFA) to a wound bed to treat the chronic wound tissue in a variety of manners, including, but not limited to, debriding debris and necrotic tissue from the wound bed, coagulation of biofilm present within the wound bed to ultimately reduce the bacterial bioburden, removal of pathogens and bacteria from the wound bed, and hemostasis via coagulating of any underlying tissue so as to prevent or stop fluid accumulation (e.g., blood from vessels), each of which promotes, stimulates, and stabilizes the wound healing process. A healthcare provider can further utilize the vacuum source for suction of any debris or excess fluid during the treatment procedure.

The devices of the present disclosure provide numerous advantages. The energy emitted from the virtual electrode of the applicator tip disrupts biological structures by creating ionic vibrations, which create friction and ultimately heat. The applicator tip is configured to desiccate the full thickness of biofilm present within a wound bed, which may be approximately 300 μm, while leaving underlying healthy tissue minimally damaged. At a cellular level, eradication of poly-microbial biofilm with a tolerable amount of healthy cell damage exposes remaining biofilm bacteria to the effect of the host immune system and antimicrobial agents. Furthermore, the device of the present disclosure is configured to provide chronic wound tissue treatment in a relative fast and efficient manner (e.g., within minutes), leading to minimal disruption in the current care path of wounds. At a clinical level, the device of the present disclosure may initially be used in conjunction with surgical or excisional debridement, as well as at the bedside on a post-procedure basis for outpatient maintenance therapy until the wound is healed. The device of the present disclosure is further useful in the pretreatment of wounds prior to excisional debridement, immediately following intraoperative surgical debridement, and as an adjunct to outpatient wound care therapy to prevent the re-establishment of biofilms. The device of the present disclosure has the potential to heal chronic, non-healing ulcers and dramatically improve patients' quality of life by avoiding many sequelae of lower extremity wounds and potential amputation.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A device for treating a chronic wound tissue, the device comprising:
a probe comprising a nonconductive elongated shaft having a proximal end and a distal end and at least one lumen extending therethrough;
a nonconductive tip extending from the distal end of the probe shaft, the nonconductive tip comprising a flexible body having a plurality of proximal ports and distal ports in communication with the at least one lumen of the probe shaft, wherein the flexible body is configured to transition from a default state to a deformed state upon application of a compression force thereto and return to the default state upon removal of the compression force therefrom; and
an electrode array comprising a plurality of independent conductive wires extending along an external surface of the nonconductive tip, each of the plurality of wires passes through an associated one of the proximal ports and through a corresponding one of the proximal ports, wherein each of the plurality of wires, or one or more sets of a combination of wires, is configured to receive an electrical current to cause activation of one or more portions of the electrode array and conduct energy for at least one of ablation and coagulation of a target portion of the chronic wound tissue when the flexible body of the nonconductive tip is in the deformed state.

2. The device of claim 1, wherein, when the flexible body of the nonconductive tip is in the default state, the electrode array is maintained a distance away from the target portion of the chronic wound tissue sufficient to prevent ablation or coagulation of the target portion.

3. The device of claim 2, wherein the flexible body comprises a distal tip portion configured to directly engage the target portion of the chronic wound tissue and maintain separation between the energy emitted from the electrode array and the target portion when the flexible body of the nonconductive tip is in the default state.

4. The device of claim 3, wherein the distal tip portion is configured to compress inwardly to decrease distance between the electrode array and the target portion of the chronic wound tissue when the flexible body of the nonconductive tip transitions from the default state to the deformed state.

5. The device of claim 1, wherein, when the flexible body of the nonconductive tip is in the deformed state, at least two of the plurality of conductive wires are positioned adjacent to the target portion of the chronic wound tissue to permit energy emitted from the electrode array to cause ablation or coagulation of the target portion.

6. The device of claim 1, wherein the flexible body of the nonconductive tip comprises a cavity in fluid communication with at least one lumen of the probe shaft and configured to receive an amount of fluid delivered from the at least one lumen.

7. The device of claim 6, wherein the device further comprises a heating element configured to heat fluid within the cavity in response to an electrical current applied thereto.

8. The device of claim 7, wherein the flexible body of the nonconductive tip is configured to transfer thermal energy from the heated fluid within the cavity to the target portion of the chronic wound tissue.

9. The device of claim 8, wherein the heating element is configured to heat the fluid to a temperature sufficient to cause necrosis of the target portion of the chronic wound tissue.

10. The device of claim 6, wherein the flexible body of the nonconductive tip further comprises one or more perforations configured to allow passage of fluid from the cavity to an external surface of the flexible body.

12. The device of claim 10, wherein energy conducted by each of the plurality of wires, or one or more sets of a combination of wires, is to be carried by fluid passing through the one or more perforations for at least one of ablation and coagulation of the target portion of the chronic wound tissue.

13. The device of claim 10, wherein the device further comprises a sensor configured to detect the presence and absence of the fluid on the external surface of the flexible body of the nonconductive tip.

14. The device of claim 6, wherein the flexible body is configured to release an amount of fluid through the one or more perforations in response to the compression force applied thereto.

15. The device of claim 1, wherein the flexible body of the nonconductive tip comprises an elastomeric material or shape memory material.

16. The device of claim 1, wherein each of the plurality of conductive wires is independent from one another such that each of the plurality of conductive wires, or one or more sets of a combination of conductive wires, is configured to independently receive an electrical current from an energy source and independently conduct energy including RF energy.

17. The device of claim 1, wherein each of the distal ports of the nonconductive tip corresponds to one proximal port such that a wire passing through corresponding distal and proximal ports extends along the length of the nonconductive tip, wherein each of the plurality of wires extends through a different distal port and each of the plurality of wires extends through a different proximal port.

18. The device of claim 1, further comprising a controller configured to selectively control supply of an electrical current to the electrode array in one or more operating modes.

19. The device of claim 19, wherein the electrode array is configured to operate in a bipolar mode, wherein a set of the plurality of conductive wires is configured to conduct RF energy.

20. The device of claim 1, wherein the probe comprises at least a second lumen extending through the elongated shaft and configured to be coupled to a vacuum source to provide suction.
